(19) United States
(12) Patent Application Publication
(10) Pub. No.: US 2003/0077679 A1
(43) Pub. Date: Apr. 24, 2003

(54) Methods for finding modulators of DYRK3 and DYRK2

(76) Inventors: Roland S. Annan, King of Prussia, PA (US); Brian M. Burns, King of Prussia, PA (US); Caretha Lee Creasy, King of Prussia, PA (US); Kathleen M. Herold, King of Prussia, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/253,349

(22) Filed: Sep. 24, 2002

Related U.S. Application Data
(60) Provisional application No. 60/326,973, filed on Oct. 4, 2001.

(51) Int. Cl. G01N 33/53; G01N 33/537; G01N 33/543; C12Q 1/42

(52) U.S. Cl. 435/7.92; 435/21

(57) ABSTRACT

Methods of finding agonists and antagonists to DYRK3 or DYRK2 are provided.

### CROSS-REFERENCE TO RELATED APPLICATIONS

This application claims priority to the earlier provisional U.S. application, Serial No. 60/326,973, which was filed on Oct. 4, 2001, the contents of which are herein incorporated by reference in their entirety.

### FIELD OF THE INVENTION

The invention relates to methods of finding agonists and antagonists of human DYRK3, murine DYRK3, and human DYRK2.

### BACKGROUND

A number of polypeptide growth factors and hormones mediate their cellular effects through a signal transduction pathway. Transduction of signals from the cell surface receptors for these ligands to intracellular effectors frequently involves phosphorylation or dephosphorylation of specific protein substrates by regulatory protein serine/threonine kinases (PSTK) and phosphatases. Serine/threonine phosphorylation is a major mediator of signal transduction in multicellular organisms. Receptor-bound, membrane-bound and intracellular PSTKs regulate cell proliferation, cell differentiation and signaling processes in many cell types. Aberrant protein serine/threonine kinase activity has been implicated or is suspected in a number of pathologies such as rheumatoid arthritis, psoriasis, septic shock, bone loss, many cancers and other proliferative diseases. Accordingly, serine/threonine kinases and the signal transduction pathways which they are part of are potential targets for drug design.

Recently, a mammalian kinase termed DYRK3 was shown to function as a suppressor of red cell development in both human and mouse bone marrow (Lord, et al., (2000) Blood 95, 2838-2846; Geiger, et al., (2001 Blood 97, 901-910). Antisense oligonucleotides directed against DYRK3 promote erythroid colony formation of bone marrow cells without affecting other colony forming units. In addition, DYRK3 is expressed predominantly in hematopoietic tissues, and within erythroid or erythropoietin (EPO)-responsive cells in particular. Together, this information suggests that inhibition of DYRK3 could serve as a treatment for anemia.

### SUMMARY OF THE INVENTION

In one aspect, the instant invention relates to a method of identifying an agonist or antagonist of human DYRK2 (SEQ ID NO:6), said method comprising the steps of:

(a) incubating a candidate compound with human DYRK2 (SEQ ID NO:6); and

(b) determining the amount of phosphorylation of human EID-1 (SEQ ID NO:4) by human DYRK2 (SEQ ID NO:6); and

(c) identifying a compound that promotes the phosphorylation human EID-1 (SEQ ID NO:4) as an agonist of human DYRK2 (SEQ ID NO:6), and a compound that decreases the phosphorylation of human EID-1 (SEQ ID NO:4) as an antagonist of human DYRK2 (SEQ ID NO:6).

In a second aspect, the instant invention relates to a method of identifying an agonist or antagonist of DYRK3, said method comprising the steps of:

(a) incubating a candidate compound with a DYRK3 selected from group consisting of: murine DYRK2 (SEQ ID NO:3) and human DYRK3 (SEQ ID NO:2); and

(b) measuring the amount of phosphorylation of human EID-1 (SEQ ID NO:4) by said DYRK3; and

(c) identifying a compound that promotes the phosphorylation of human EID-1 (SEQ ID NO:4) as an agonist of said DYRK3 selected from group consisting of: murine DYRK2 (SEQ ID NO:3) and human DYRK3 (SEQ ID NO:2), and a compound that decreases the phosphorylation of human EID-1 (SEQ ID NO:4) as an antagonist of said DYRK3 selected from group consisting of: murine DYRK2 (SEQ ID NO:3) and human DYRK3 (SEQ ID NO:2).

In a third aspect, the instant invention relates to the above-recited method, wherein the DYRK3 is human DYRK3 comprising the amino acid sequence of residues 142-526 of SEQ ID NO:2.

In a fourth aspect, the instant invention relates to any of the above-recited methods, wherein the amount of phosphorylation of human EID-1 (SEQ ID NO:4) is measured using an antibody specific to phosphorylated human EID-1 (SEQ ID NO:4).

In a fifth aspect, the instant invention relates to any of the above-recited methods, wherein the human EID-1 is phosphorylated on serine 125 of human EID-1 (SEQ ID NO:4).

In a sixth aspect, the instant invention relates to any of the above-recited methods, wherein the amount of phosphorylation of human EID-1 (SEQ ID NO:4) is measured by Liquid Chromatography-Electrospray Tandem Mass Spectrometry (LC-ES MS/MS).

In a seventh aspect, the instant invention relates to any of the above-recited methods, wherein the amount of phosphorylation of human EID-1 (SEQ ID NO:4) is measured using a labeled ATP.

In an eighth aspect, the instant invention relates to any of the above-recited methods, wherein the labeled ATP is P³³ labeled ATP.

### BRIEF DESCRIPTION OF THE DRAWINGS

FIG. 1 shows the nucleotide and amino acid sequences of human DYRK3 (SEQ NOs:1 and 2, respectively).

FIG. 2 shows the deduced amino acid sequence of murine DYRK3 (SEQ NO:3).

FIG. 3 shows the amino acid and nucleotide sequences of human EID-1 (SEQ NOs:4 and 5, respectively).

FIG. 4 shows the deduced amino acid sequence of human DYRK2 (SEQ ID NO:6).

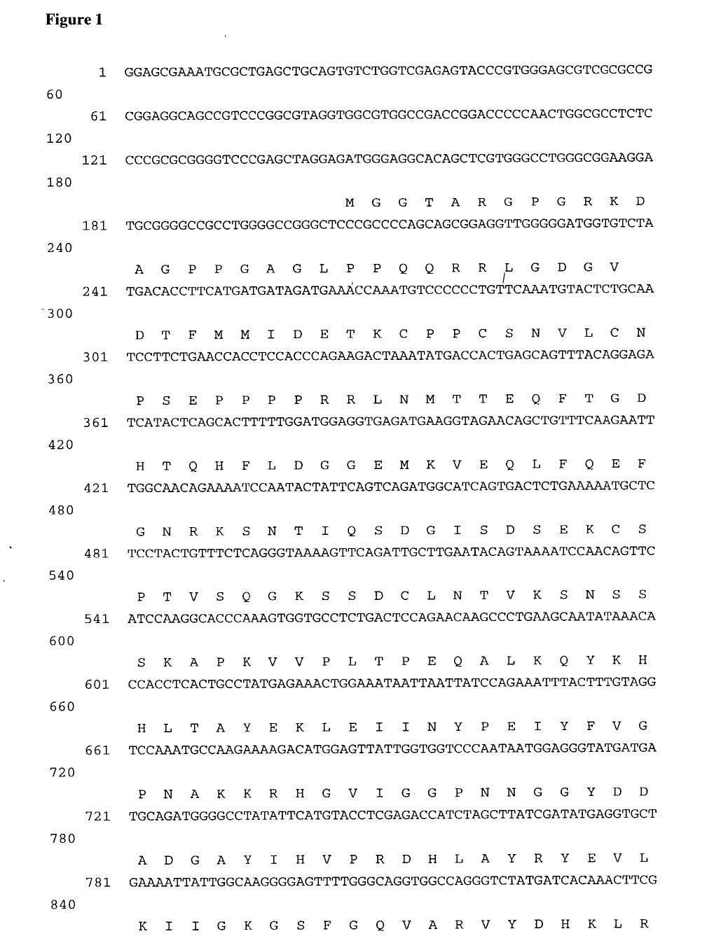

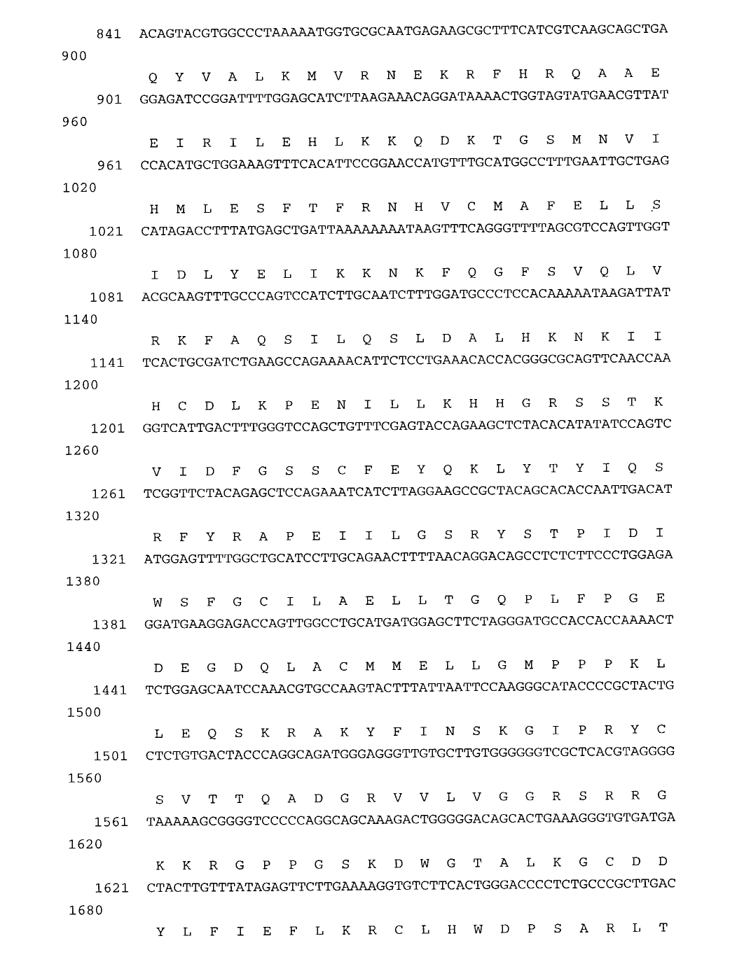

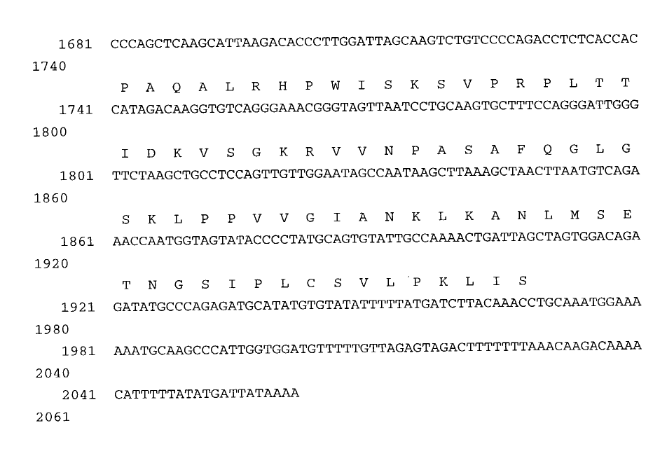

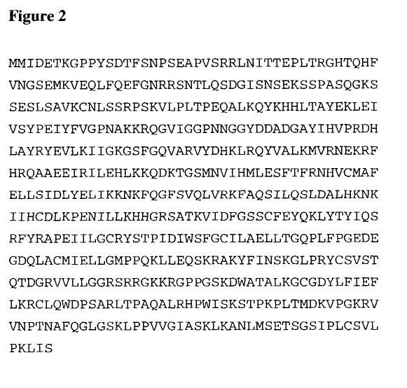

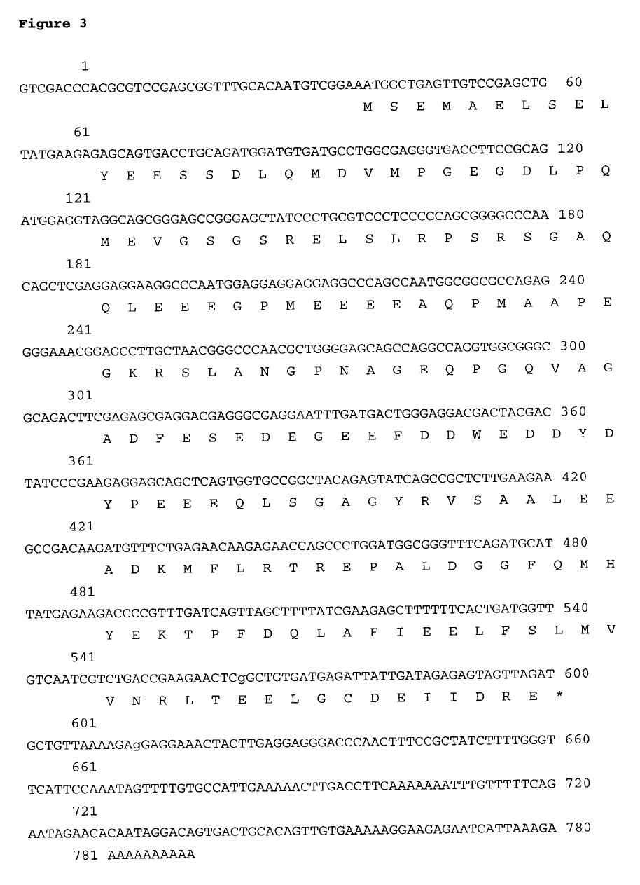

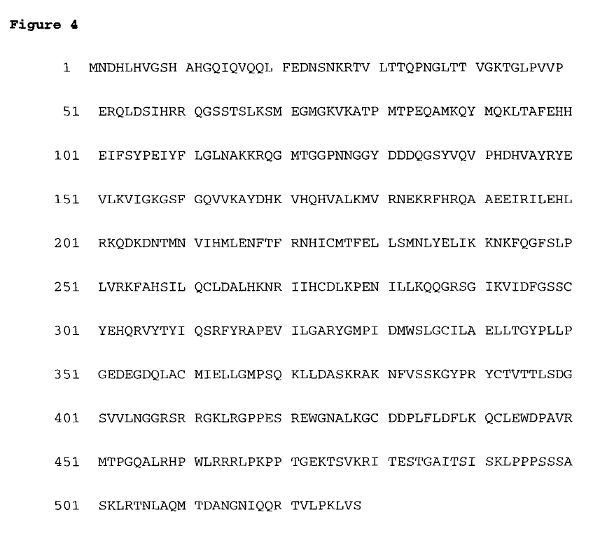

### DETAILED DESCRIPTION OF THE INVENTION

Given DYRK2 and DYRK3's potential involvement in erythropoiesis, it is of interest to discover activators and substrates therefor. As disclosed herein, human DYRK3 (hDYRK3) was used as the bait in a yeast two-hybrid screen (Fields, et al., (1989) Nature 340, 245-247) to identify human EID-1 as a protein which binds to and is an in vitro substrate of DYRK3. Human EID-1 was previously identified as an Rb and p300-binding protein, and inhibitor of differentiation [Miyake, et al., (200) Mol. Cell. Biol. 20, 8889-8902; MacLellan, et al., (2000) Mol. Cell. Biol. 20, 8903-8915]. Inhibitors of DYRK2 or DYRK3, in turn, decrease the phosphorylation of human EID-1 (SEQ ID NO:4). Compounds identified that inhibit the phosphorylation of human EID-1 may be useful as therapeutics for erythropoiesis.

The observation discussed below in Example 3 that either DYRK2 or DYRK3 can phosphorylate human EID-1 can be exploited to configure a screening method for identifying a compound which modulates the activity of DYRK3 or DYRK2. More particularly, the method comprises the step of incubating a candidate compound with DYRK3 or DYRK2, and determining the amount of phosphorylation on EID-1 by DYRK3 or DYRK2, whereby a compound which promotes the phosphorylation is an agonist of DYRK3 or DYRK2, and a compound which decreases the phosphorylation is an inhibitor of DYRK3 or DYRK2.

In a more preferred method, DYRK3 is a polypeptide of SEQ ID NO:2 or 3 or a polypeptide with amino acid sequence 142-526 of SEQ ID NO:2, and EID-1 is a polypeptide of SEQ ID NO:4; and DYRK2 is a polypeptide of SEQ ID NO:6. Yet in another preferred method, the amount of phosphorylation is measured using an antibody specific to phosphorylated EID-1 or using a labeled ATP, such as P³²- or P³³-ATP.

Using this data generated from the mass spectrometry, discussed below in Example 4, tools such as phosphospecific antibodies directed to a peptide derived from hEID-1 (SEQ ID NO:4) encompassing serine 125, or variants thereof, can be used to identify the phosphorylation state of this residue in cells or tissues expressing hDYRK3 (SEQ ID NO:2). In addition, one could evaluate the phosphorylation state of hEID-1 (SEQ ID NO:4) in cells or tissues treated with hDYRK3 agonists or inhibitors.

Definitions

The following definitions are provided to facilitate understanding of certain terms used frequently herein

“DYRK2” (or DYRK2 polypeptides) generally refers to human DYRK2 polypeptide having the amino acid sequence set forth in SEQ ID NO:6 or an allelic variant thereof.

“DYRK3” (or DYRK3 polypeptides) generally refers to human DYRK3 polypeptide having the amino acid sequence set forth in SEQ ID NO:2, or an allelic variant thereof, or to murine DYRK3 having the amino acid sequence set forth in SEQ ID NO:3.

“EID-1” (or EID-1 polypeptides) refers to human EID-1 polypeptide having the amino acid sequence set forth in SEQ ID NO:4 or an allelic variant thereof.

Polypeptides of the present invention also include variants of the aforementioned polypeptides. Such polypeptides vary from the reference polypeptide by insertions, deletions, and substitutions that may be conservative or non-conservative, or any combination thereof. Particularly preferred variants are those in which several, for instance from 50 to 30, from 30 to 20, from 20 to 10, from 10 to 5, from 5 to 3, from 3 to 2, from 2 to 1 or 1 amino acids are inserted, substituted, or deleted, in any combination.

Preferred fragments of polypeptides of the present invention include an isolated polypeptide comprising an amino acid sequence having at least 30, 50 or 100 contiguous amino acids from the amino acid sequence of SEQ ID NOs:2, 3, 4, or 6, or an isolated polypeptide comprising an amino acid sequence having at least 30, 50 or 100 contiguous amino acids truncated or deleted from the amino acid sequence of SEQ ID NOs:2, 3, 4, or 6. Preferred fragments are biologically active fragments that mediate the biological activity of DYRK2, DYRK3, or EID-1, including those with a similar activity or an improved activity, or with a decreased undesirable activity. For example, preferred fragments for DYRK3 include a polypeptide which has the amino acid sequence 142-526 of SEQ ID NO:2, which retains a catalytic activity.

Polypeptides of the present invention can be prepared in any suitable manner, for instance by isolation from naturally occurring sources, from genetically engineered host cells comprising expression systems or by chemical synthesis, using for instance automated peptide synthesizers, or a combination of such methods. Means for preparing such polypeptides are well understood in the art.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living.

“Polypeptide” refers to any polypeptide comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques that are well known in the art. Such modifications are well-described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (see, for instance, Proteins—Structure and Molecular Properties, 2nd Ed., T. E. Creighton, W. H. Freeman and Company, New York, 1993; Wold, F., Post-translational Protein Modifications: Perspectives and Prospects, 1-12, in Post-translational Covalent Modification of Proteins, B. C. Johnson, Ed., Academic Press, New York, 1983; Seifter, et al., “Analysis for protein modifications and nonprotein cofactors”, Meth Enzymol, 182, 626-646, 1990, and Rattan, et al., “Protein Synthesis: Post-translational Modifications and Aging”, Ann NY Acad Sci, 663, 48-62, 1992).

“Fragment” of a polypeptide sequence refers to a polypeptide sequence that is shorter than the reference sequence, and preferably retains essentially the same biological function or activity as the reference polypeptide.

“Variant” refers to a polypeptide that differs from a reference polypeptide, and preferably retains the essential properties thereof. Generally, alterations are limited so that the sequences of the reference polypeptide and the variant are closely similar overall and, in many regions, identical. A variant and reference polypeptide may differ in amino acid sequence by one or more substitutions, insertions, or deletions in any combination. A substituted or inserted amino acid residue may or may not be one encoded by the genetic code. Typical conservative substitutions include Gly, Ala; Val, Ile, Leu; Asp, Glu; Asn, Gln; Ser, Thr; Lys, Arg; and Phe and Tyr. A variant of a polypeptide may be naturally occurring such as an allele, or it may be a variant that is not known to occur naturally. Non-naturally occurring variants of polypeptides may be made by mutagenesis techniques or by direct synthesis. Also included as variants are polypeptides having one or more post-translational modifications, for instance glycosylation, phosphorylation, methylation, ADP ribosylation and the like. Embodiments include methylation of the N-terminal amino acid, phosphorylations of tyrosines, serines, and threonines and modification of C-terminal glycines.

“Identity” reflects a relationship between two or more polypeptide sequences, determined by comparing the sequences. In general, identity refers to an exact amino acid to amino acid correspondence of the two polypeptide sequences, respectively, over the length of the sequences being compared.

“% Identity”—For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

Methods for comparing the identity of two or more sequences are well known in the art. Thus for instance, programs available in the Wisconsin Sequence Analysis Package, version 9.1 (Devereux J, et al., Nucleic Acids Res, 12, 387-395, 1984, available from Genetics Computer Group, Madison, Wis., USA), BESTFIT and GAP, may be used to determine the % identity between two polypeptide sequences. BESTFIT uses the “local homology” algorithm of Smith and Waterman (J Mol Biol, 147,195-197, 1981, Advances in Applied Mathematics, 2, 482-489, 1981) and finds the best single region of similarity between two sequences. BESTFIT is more suited to comparing two polypeptide sequences that are dissimilar in length, the program assuming that the shorter sequence represents a portion of the longer. In comparison, GAP aligns two sequences, finding a “maximum similarity”, according to the algorithm of Neddleman and Wunsch (J Mol Biol, 48, 443-453, 1970). GAP is more suited to comparing sequences that are approximately the same length and an alignment is expected over the entire length. Preferably, the parameters “Gap Weight” and “Length Weight” used in each program are 12 and 4 for polypeptide sequences, respectively. Preferably, % identities are determined when the two sequences being compared are optimally aligned.

Other programs for determining identity and/or similarity between sequences are also known in the art, for instance the BLAST family of programs (Altschul S. F., et al., J Mol Biol, 215, 403-410, 1990, Altschul, S. F., et al., Nucleic Acids Res., 25:389-3402, 1997, available from the National Center for Biotechnology Information (NCBI), Bethesda, Md., USA and accessible through the home page of the NCBI at www.ncbi.nlm.nih.gov) and FASTA (Pearson W R, Methods in Enzymology, 183, 63-99, 1990; Pearson W R and Lipman D J, Proc Nat Acad Sci USA, 85, 2444-2448,1988, available as part of the Wisconsin Sequence Analysis Package).

Preferably, the BLOSUM62 amino acid substitution matrix (Henikoff S and Henikoff J G, Proc. Nat. Acad Sci. USA, 89, 10915-10919, 1992) is used in polypeptide sequence comparisons including where nucleotide sequences are first translated into amino acid sequences before comparison.

Preferably, the program BESTFIT is used to determine the % identity of a query polypeptide sequence with respect to a reference polypeptide sequence, the query and the reference sequence being optimally aligned and the parameters of the program set at the default value, as hereinbefore described.

“Identity Index” is a measure of sequence relatedness which may be used to compare a candidate sequence (polypeptide) and a reference sequence. Thus, for instance a candidate polypeptide sequence having, for example, an Identity Index of 0.95 compared to a reference polypeptide sequence is identical to the reference sequence except that the polypeptide sequence may include an average of up to five differences per each 100 amino acids of the reference sequence. Such differences are selected from the group consisting of at least one amino acid deletion, substitution, including conservative and non-conservative substitution, or insertion. These differences may occur at the amino- or carboxy-terminal positions of the reference polypeptide sequence or anywhere between these terminal positions, interspersed either individually among the amino acids in the reference sequence or in one or more contiguous groups within the reference sequence. In other words, to obtain a polypeptide sequence having an Identity Index of 0.95 compared to a reference polypeptide sequence, an average of up to 5 in every 100 of the amino acids in the reference sequence may be deleted, substituted or inserted, or any combination thereof, as hereinbefore described. The same applies mutatis mutandis for other values of the Identity Index, for instance 0.96, 0.97, 0.98 and 0.99.

The relationship between the number of amino acid differences and the Identity Index may be expressed in the following equation:

nₐ≦xₐ−(xₐ·I),

in which:

nₐ is the number of amino acid differences,

xₐ is the total number of amino acids in SEQ ID NO:2, 3, or 4,

I is the Identity Index,

· is the symbol for the multiplication operator, and

in which any non-integer product of xₐ and I is rounded down to the nearest integer prior to subtracting it from xₐ.

### BIOLOGICAL METHODS/EXAMPLES

### Example 1

Full-length human DYRK3 cDNA (SEQ ID NO: 1) was PCR amplified and cloned into pEG202 (Golemis, et al., in Current Protocols in Molecular Biology, John Wiley & Sons, New York (1994) using traditional molecular biology methods. The resulting plasmid, pEG202-DYRK3, encoded a fusion protein in which the DNA binding protein, LexA, was fused in-frame to DYRK3. The parent vector, pEG202, was a yeast expression vector which uses the alcohol dehydrogenase (ADH1) promoter to express the LexA fusion proteins and HIS3 as the selectable marker. All procedures, plasmids and strains used in the two-hybrid screen have been described in detail by Golemis, et al., supra. Yeast strain EGY48B (MATα, trp1, his3, ura3, 6ops-LEU2) was cotransformed with the plasmids pEG202-DYRK3 and pMW107. Transformants were selected using complete minimal media lacking uracil and histidine. The plasmid pPW107 is a yeast expression vector in which eight LexA operator sites are located upstream of a minimal GAL1 promoter which drives the expression of the LacZ gene, and URA3 as a selectable marker. Synthesis of the full length LexA-DYRK3 fusion was confirmed by Western blot analysis of yeast extracts using polyclonal antisera directed against LexA. It was confirmed that the LexA-DYRK3 fusion alone was unable to activate the LEU2 nor LacZ reporter strains.

Strain EGY48 ((MATα, trp1, his3, ura3, 6ops-LEU2) was transformed with a human fetal liver cDNA library in plasmid pJG4-5 using a library scale transformation protocol. This library plasmid contains the TRP1 selectable marker and allows the expression of cDNAs as fusions (AD fusions) to a cassette containing the SV40 nuclear localization sequence, the acid blob B42, and the hemagglutinin epitope tag. See Gyuris et al., Cell 75, 791-803 (1993). Expression of this fusion is under control of the galactose inducible promoter GAL1. Transformation reactions were plated onto complete minimal media lacking tryptophan. Approximately 4.5×10⁶ individual transformants were obtained, pooled and frozen. To ensure that each primary colony containing a library plasmid was mated, 2×10⁷ cells (approximately 3 times the number of individual transformants) were mixed with 2×10⁸ cells containing pEG202-DYRK3 and pMW107. The mixed cells were incubated on a nitrocellulose/YPD plate overnight at 30° C. to allow mating. Cells were washed off the nitrocellulose and plated onto minimal media lacking uracil, histidine, tryptophan and leucine with galactose/raffinose as the carbon source to induce expression of AD fusions. Colonies arising after 3 and 4 days of growth at 30° C. were picked to complete minimal media lacking uracil, histidine and tryptophan. Colonies containing potential interacting fusion proteins were then tested for galactose dependence and LacZ expression. Those isolates which activated both the LEU2 and LacZ reporters in a galactose dependent fashion were considered positive and pursued further.

Sequence analysis and subsequent GenBank searches using the BLASTX and BLATSN algorithms with the cDNA sequence or with the deduced amino acid sequence indicated that one of the AD fusion plasmids (isolate YFL11) encoded a protein identical retinoblastoma protein-associated protein (AF109873). Subsequent to this finding Miyake, et al. and MacLellan, et al. [MCB (2000 20, 8889-8902; MCB (2000) 20, 8903-8915] identified YFL11 as a protein which they term human EID-1 (SEQ ID NO:4).

### Example 2

To determine whether DYRK3 and EID-1 interact in mammalian cells, murine DYRK3 (SEQ ID NO:3) was engineered for mammalian expression with an N-terminal cmyc6 tag in pCDNA3.1 (Invitrogen) and human EID-1 (SEQ ID NO:4) was engineered with an N-terminal V5-HIS6 tag in p423CDN, using standard molecular techniques. HEK293 cells were transfected with each construct together or singly using FUGENE6 transfection reagent (Roche Molecular Biochemicals). Two days after the transfection, the cells were washed twice in phosphate-buffered saline, and lysed in NP-40 lysis buffer (50 mM Tris-HCl, pH7.5, 150 mM NaCl, 10% glycerol, 1% Nonidet P-40) supplemented with 1×Complete protease inhibitor cocktail (Roche Molecular Biochemicals), 50 mM NaF, 10 mM β-glycerol phosphate, and 1 mM sodium vanadate. Lysates were clarified via centrifugation at 12,000×g for 5 min, and protein concentration determined using BCA protein reagent (Pierce). To immunoprecipitate, 1 ug of anti-V5 antibodies (Invitrogen) and 250 ul of IP buffer [50 mM Tris-HCl, pH7.5, 150 mM NaCl, 10% glycerol, 0.1% Nonidet P-40) supplemented with 1×Complete protease inhibitor cocktail (Roche Molecular Biochemicals)] was added to 300 ug of protein lysate and the reaction mixture incubated at 4° C. overnight. Protein A-Sepharose beads (100 ul of 50% slurry, Life Technologies) were added and incubation continued for 45 min. The beads were washed three times in IP buffer and resuspended in 40 ul 2×SDS sample buffer+5 ul reducing agent (Novex). The material was then separated via SDS page, transferred to nitrocellulose and subjected to western blot analysis with anti-cmyc or anti-V5 antibodies using standard conditions. Western blot analysis indicated that V5-EID-1 was immunoprecipitated from HEK293 cells transfected with V5-EID-1 and that cmyc-mDYRK3 is immunoprecipitated only when V5-EID-1 was present. This data, together with the yeast two-hybrid data, indicate that DYRK3 and EID-1 proteins interact.

### Example 3

To determine if human EID-1 can serve as a substrate for DYRK3 or DYRK2, HEK 293 cells were transfected with p423CDN-V5-EID-1, and V5-EID-1 was immunoprecipitated as described in Example 2. The immunoprecipitated material was then subjected to western blot analysis to confirm the immunoprecipitation and an in vitro kinase reaction with purified DYRK3 or DYRK2 protein. To purify DYRK3, the catalytic domain (amino acids 142-526 from SEQ ID NO:2) was engineered for expression from a Baculovirus expression system with an N-terminal GST-tag and C-terminal HIS6-tag. Following infection of SF9 cells with virus, protein lysate was first passed over a glutathione-sepharose column followed by elution with 10 mM glutathione. Partially purified DYRK3 was then passed over a nickel column and eluted with 250 mM imidazole. To purify DYRK2, the protein with amino acid of SEQ ID NO:6 was engineered for expression from a Baculovirus expression system with an N-terminal HIS6-tag. Following infection of SF9 cells with virus, protein lysate was passed over a nickel column and eluted with 250 mM imidazole. To perform the in vitro kinase assay, immunoprecipitated V5-EID-1 or vector alone was incubated with kinase buffer containing 100 ng DYRK3 or 50 ng DYRK2 in 25 mM HEPES, pH 7.5, 10 mM MgCl₂, 10 mM β-glycerophosphate, 0.2 mM sodium orthovanadate, 50 uM adenosine triphosphate (ATP) and 5 uCi γ-³³P-ATP at 37° C. for 15 minutes. The reaction was terminated by the addition of sample buffer and boiling followed by electrophoresis and exposure to a Phosphorimager Screen (Molecular Dynamics, Sunnyvale, Calif.). There is a radioactive band that migrates at the same position as the EID-1 protein in the western blot while this band is absent in the vector alone lane in both the kinase reaction and western blot. This data demonstrates that EID-1 is phosphorylated by DYRK3 and DYRK2 in vitro.

In conclusion, the observation that either DYRK2 or DYRK3 can phosphorylate human EID-1 can be exploited to configure a screening method for identifying a compound which modulates the activity of DYRK3 or DYRK2. More particularly, the method comprises the step of incubating a candidate compound with DYRK3 or DYRK2, and determining the amount of phosphorylation on EID-1 by DYRK3 or DYRK2, whereby a compound which promotes the phosphorylation is an agonist of DYRK3 or DYRK2, and a compound which decreases the phosphorylation is an inhibitor of DYRK3 or DYRK2.

In a more preferred method, DYRK3 is a polypeptide of SEQ ID NO:2 or 3 or a polypeptide with amino acid sequence 142-526 of SEQ ID NO:2, and EID-1 is a polypeptide of SEQ ID NO: 4; and DYRK2 is a polypeptide of SEQ ID NO:6. Yet in another preferred method, the amount of phosphorylation is measured using an antibody specific to phosphorylated EID-1 or using a labeled ATP, such as P³²- or P³³-ATP.

### Example 4

Human EID-1 (SEQ ID NO:4) was expressed in E. coli as an N-terminal HIS6 fusion and the protein purified by traditional methods known to those skilled in the art. To identify the site(s) on hEID-1 that are phosphorylated by hDYRK3 (SEQ ID NO: 2), an in vitro kinase assay was performed as follows: 10 μg purified EID-1 was incubated with kinase buffer containing 600 ng recombinant hDYRK3 in 44 mM HEPES, pH 7.5, 20 mM MgCl₂, 7 mM β-glycerophosphate, 0.14 mM sodium orthovanadate, 70 μM EDTA, 0.0018% Tween-20, 9.6 mM β-mercaptoethanol, 50 μM adenosine triphosphate (ATP) with or without 5 μCi g-33P-ATP at room temperature for 1 hr in a 100 μl reaction. The reaction was terminated by the addition of 0.1% TFA followed by electrophoresis and exposure to a Phosphorimager Screen (Molecular Dynamics, Sunnyvale, Calif.). In the reaction utilizing radioactivity, there was a radioactive band that migrated at the same position as the EID-1 protein following electrophoresis. The corresponding region in the lane that did not contain radioactivity was cut out of the gel and digested with Asp-N followed by Liquid Chromatography-Electrospray Tandem Mass Spectrometry (LC-ES MS/MS) to detect the site of phosphorylation. LC-ES MS/MS is a method that is well understood by those of skill in the art of mass spectrometry. A single phosphorylation site was identified on serine 125 of hEID-1 in the peptide DYPEEEQLSGAGYRVSAALEEA (SEQ ID NO:7). Using this information, tools such as phosphospecific antibodies directed to a peptide derived from hEID-1 (SEQ ID NO:4) encompassing serine 125, or variants thereof, can be used to identify the phosphorylation state of this residue in cells or tissues expressing hDYRK3 (SEQ ID NO:2). In addition, one could evaluate the phosphorylation state of hEID-1 (SEQ ID NO:4) in cells or tissues treated with hDYRK3 agonists or inhibitors.

All publications including, but not limited to, patents and patent applications, cited in this specification, are herein incorporated by reference as if each individual publication were specifically and individually indicated to be incorporated by reference herein as though fully set forth.

The above description fully discloses the invention, including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the examples provided herein are to be construed as merely illustrative and are not a limitation of the scope of the present invention in any way. The embodiments of the invention in which an exclusive property or privilege is claimed are defined as follows.

We claim:

1. A method of identifying an agonist or antagonist of human DYRK2 (SEQ ID NO:6), said method comprising the steps of:
(a) incubating a candidate compound with human DYRK2 (SEQ ID NO:6); and
(b) determining the amount of phosphorylation of human EID-1 (SEQ ID NO:4) by human DYRK2 (SEQ ID NO:6); and
(c) identifying a compound that promotes the phosphorylation human EID-1 (SEQ ID NO:4) as an agonist of human DYRK2 (SEQ ID NO:6), and a compound that decreases the phosphorylation of human EID-1 (SEQ ID NO:4) as an antagonist of human DYRK2 (SEQ ID NO:6).

2. A method of identifying an agonist or antagonist of DYRK3, said method comprising the steps of:
(a) incubating a candidate compound with a DYRK3 selected from group consisting of: murine DYRK2 (SEQ ID NO:3) and human DYRK3 (SEQ ID NO:2); and
(b) measuring the amount of phosphorylation of human EID-1 (SEQ ID NO:4) by said DYRK3; and
(c) identifying a compound that promotes the phosphorylation of human EID-1 (SEQ ID NO:4) as an agonist of said DYRK3 selected from group consisting of: murine DYRK2 (SEQ ID NO:3) and human DYRK3 (SEQ ID NO:2), and a compound that decreases the phosphorylation of human EID-1 (SEQ ID NO:4) as an antagonist of said DYRK3 selected from group consisting of: murine DYRK2 (SEQ ID NO:3) and human DYRK3 (SEQ ID NO:2).

3. The method as claimed in claim 2, wherein the DYRK3 is human DYRK3 comprising the amino acid sequence of residues 142-526 of SEQ ID NO:2.

4. The method as claimed in claim 1, wherein the amount of phosphorylation of human EID-1 (SEQ ID NO:4) is measured using an antibody specific to phosphorylated human EID-1 (SEQ ID NO:4).

5. The method as claimed in claim 4, wherein the human EID-1 is phosphorylated on serine 125 of human EID-1 (SEQ ID NO:4).

6. The method as claimed in claim 4, wherein the amount of phosphorylation of human EID-1 (SEQ ID NO:4) is measured by Liquid Chromatography-Electrospray Tandem Mass Spectrometry (LC-ES MS/MS).

7. The method as claimed in claim 1, wherein the amount of phosphorylation of human EID-1 (SEQ ID NO:4) is measured using a labeled ATP.

8. The method as claimed in claim 7, wherein the labeled ATP is P³³ labeled ATP.

9. The method of claim 2, wherein the amount of phosphorylation of human EID-1 (SEQ ID NO:4) is measured using an antibody specific to phosphorylated human EID-1 (SEQ ID NO:4).

10. The method as claimed in claim 9, wherein the human EID-1 is phosphorylated on serine 125 of human EID-1 (SEQ ID NO:4).

11. The method as claimed in claim 9, wherein the amount of phosphorylation of human EID-1 (SEQ ID NO:4) is measured by Liquid Chromatography-Electrospray Tandem Mass Spectrometry (LC-ES MS/MS).

12. The method of claim 3, wherein the amount of phosphorylation of human EID-1 (SEQ ID NO:4) is measured using an antibody specific to phosphorylated human EID-1 (SEQ ID NO:4).

13. The method as claimed in claim 12, wherein the human EID-1 is phosphorylated on serine 125 of human EID-1 (SEQ ID NO:4).

14. The method as claimed in claim 12, wherein the amount of phosphorylation of human EID-1 (SEQ ID NO:4) is measured by Liquid Chromatography-Electrospray Tandem Mass Spectrometry (LC-ES MS/MS).

15. The method as claimed in claim 3, wherein the amount of phosphorylation of human EID-1 (SEQ ID NO:4) is measured using a labeled ATP.

16. The method as claimed in claim 15, wherein the labeled ATP is P³³ labeled ATP.


SEQUENCE LISTING (7 sequences)

SEQ ID NO: 1 - DNA, 2061 bp, Homo sapiens
ggagcgaaatgcgctgagctgcagtgtctggtcgagagtacccgtgggagcgtcgcgccgcggaggcagccgtcccggcgtaggtggcgtggccgaccggacccccaactggcgcctctccccgcgcggggtcccgagctaggagatgggaggcacagctcgtgggcctgggcggaaggatgcggggccgcctggggccgggctcccgccccagcagcggaggttgggggatggtgtctatgacaccttcatgatgatagatgaaaccaaatgtcccccctgttcaaatgtactctgcaatccttctgaaccacctccacccagaagactaaatatgaccactgagcagtttacaggagatcatactcagcactttttggatggaggtgagatgaaggtagaacagctgtttcaagaatttggcaacagaaaatccaatactattcagtcagatggcatcagtgactctgaaaaatgctctcctactgtttctcagggtaaaagttcagattgcttgaatacagtaaaatccaacagttcatccaaggcacccaaagtggtgcctctgactccagaacaagccctgaagcaatataaacaccacctcactgcctatgagaaactggaaataattaattatccagaaatttactttgtaggtccaaatgccaagaaaagacatggagttattggtggtcccaataatggagggtatgatgatgcagatggggcctatattcatgtacctcgagaccatctagcttatcgatatgaggtgctgaaaattattggcaaggggagttttgggcaggtggccagggtctatgatcacaaacttcgacagtacgtggccctaaaaatggtgcgcaatgagaagcgctttcatcgtcaagcagctgaggagatccggattttggagcatcttaagaaacaggataaaactggtagtatgaacgttatccacatgctggaaagtttcacattccggaaccatgtttgcatggcctttgaattgctgagcatagacctttatgagctgattaaaaaaaataagtttcagggttttagcgtccagttggtacgcaagtttgcccagtccatcttgcaatctttggatgccctccacaaaaataagattattcactgcgatctgaagccagaaaacattctcctgaaacaccacgggcgcagttcaaccaaggtcattgactttgggtccagctgtttcgagtaccagaagctctacacatatatccagtctcggttctacagagctccagaaatcatcttaggaagccgctacagcacaccaattgacatatggagttttggctgcatccttgcagaacttttaacaggacagcctctcttccctggagaggatgaaggagaccagttggcctgcatgatggagcttctagggatgccaccaccaaaacttctggagcaatccaaacgtgccaagtactttattaattccaagggcataccccgctactgctctgtgactacccaggcagatgggagggttgtgcttgtggggggtcgctcacgtaggggtaaaaagcggggtcccccaggcagcaaagactgggggacagcactgaaagggtgtgatgactacttgtttatagagttcttgaaaaggtgtcttcactgggacccctctgcccgcttgaccccagctcaagcattaagacacccttggattagcaagtctgtccccagacctctcaccaccatagacaaggtgtcagggaaacgggtagttaatcctgcaagtgctttccagggattgggttctaagctgcctccagttgttggaatagccaataagcttaaagctaacttaatgtcagaaaccaatggtagtatacccctatgcagtgtattgccaaaactgattagctagtggacagagatatgcccagagatgcatatgtgtatatttttatgatcttacaaacctgcaaatggaaaaaatgcaagcccattggtggatgtttttgttagagtagactttttttaaacaagacaaaacatttttatatgattataaaa

SEQ ID NO: 2 - protein, 587 aa, Homo sapiens
MGGTARGPGRKDAGPPGAGLPPQQRRLGDGVDTFMMIDETKCPPCSNVLCNPSEPPPPRRLNMTTEQFTGDHTQHFLDGGEMKVEQLFQEFGNRKSNTIQSDGISDSEKCSPTVSQGKSSDCLNTVKSNSSSKAPKVVPLTPEQALKQYKHHLTAYEKLEIINYPEIYFVGPNAKKRHGVIGGPNNGGYDDADGAYIHVPRDHLAYRYEVLKIIGKGSFGQVARVYDHKLRQYVALKMVRNEKRFHRQAAEEIRILEHLKKQDKTGSMNVIHMLESFTFRNHVCMAFELLSIDLYELIKKNKFQGFSVQLVRKFAQSILQSLDALHKNKIIHCDLKPENILLKHHGRSSTKVIDFGSSCFEYQKLYTYIQSRFYRAPEIILGSRYSTPIDIWSFGCILAELLTGQPLFPGEDEGDQLACMMELLGMPPPKLLEQSKRAKYFINSKGIPRYCSVTTQADGRVVLVGGRSRRGKKRGPPGSKDWGTALKGCDDYLFIEFLKRCLHWDPSARLTPAQALRHPWISKSVPRPLTTIDKVSGKRVVNPASAFQGLGSKLPPVVGIANKLKANLMSETNGSIPLCSVLPKLIS

SEQ ID NO: 3 - protein, 551 aa, Mus musculus
MMIDETKGPPYSDTFSNPSEAPVSRRLNITTEPLTRGHTQHFVNGSEMKVEQLFQEFGNRRSNTLQSDGISNSEKSSPASQGKSSESLSAVKCNLSSRPSKVLPLTPEQALKQYKHHLTAYEKLEIVSYPEIYFVGPNAKKRQGVIGGPNNGGYDDADGAYIHVPRDHLAYRYEVLKIIGKGSFGQVARVYDHKLRQYVALKMVRNEKRFHRQAAEEIRILEHLKKQDKTGSMNVIHMLESFTFRNHVCMAFELLSIDLYELIKKNKFQGFSVQLVRKFAQSILQSLDALHKNKIIHCDLKPENILLKHHGRSATKVIDFGSSCFEYQKLYTYIQSRFYRAPEIILGCRYSTPIDIWSFGCILAELLTGQPLFPGEDEGDQLACMIELLGMPPQKLLEQSKRAKYFINSKGLPRYCSVSTQTDGRVVLLGGRSRRGKKRGPPGSKDWATALKGCGDYLFIEFLKRCLQWDPSARLTPAQALRHPWISKSTPKPLTMDKVPGKRVVNPTNAFQGLGSKLPPVVGIASKLKANLMSETSGSIPLCSVLPKLIS

SEQ ID NO: 4 - protein, 187 aa, Homo sapiens
MSEMAELSELYEESSDLQMDVMPGEGDLPQMEVGSGSRELSLRPSRSGAQQLEEEGPMEEEEAQPMAAPEGKRSLANGPNAGEQPGQVAGADFESEDEGEEFDDWEDDYDYPEEEQLSGAGYRVSAALEEADKMFLRTREPALDGGFQMHYEKTPFDQLAFIEELFSLMVVNRLTEELGCDEIIDRE

SEQ ID NO: 5 - DNA, 790 bp, Homo sapiens
gtcgacccacgcgtccgagcggtttgcacaatgtcggaaatggctgagttgtccgagctgtatgaagagagcagtgacctgcagatggatgtgatgcctggcgagggtgaccttccgcagatggaggtaggcagcgggagccgggagctatccctgcgtccctcccgcagcggggcccaacagctcgaggaggaaggcccaatggaggaggaggaggcccagccaatggcggcgccagaggggaaacggagccttgctaacgggcccaacgctggggagcagccaggccaggtggcgggcgcagacttcgagagcgaggacgagggcgaggaatttgatgactgggaggacgactacgactatcccgaagaggagcagctcagtggtgccggctacagagtatcagccgctcttgaagaagccgacaagatgtttctgagaacaagagaaccagccctggatggcgggtttcagatgcattatgagaagaccccgtttgatcagttagcttttatcgaagagcttttttcactgatggttgtcaatcgtctgaccgaagaactcggctgtgatgagattattgatagagagtagttagatgctgttaaaagaggaggaaactacttgaggagggacccaactttccgctatcttttgggttcattccaaatagttttgtgccattgaaaaacttgaccttcaaaaaaatttgtttttcagaatagaacacaataggacagtgactgcacagttgtgaaaaaggaagagaatcattaaagaaaaaaaaaaa

SEQ ID NO: 6 - protein, 528 aa, Homo sapiens
MNDHLHVGSHAHGQIQVQQLFEDNSNKRTVLTTQPNGLTTVGKTGLPVVPERQLDSIHRRQGSSTSLKSMEGMGKVKATPMTPEQAMKQYMQKLTAFEHHEIFSYPEIYFLGLNAKKRQGMTGGPNNGGYDDDQGSYVQVPHDHVAYRYEVLKVIGKGSFGQVVKAYDHKVHQHVALKMVRNEKRFHRQAAEEIRILEHLRKQDKDNTMNVIHMLENFTFRNHICMTFELLSMNLYELIKKNKFQGFSLPLVRKFAHSILQCLDALHKNRIIHCDLKPENILLKQQGRSGIKVIDFGSSCYEHQRVYTYIQSRFYRAPEVILGARYGMPIDMWSLGCILAELLTGYPLLPGEDEGDQLACMIELLGMPSQKLLDASKRAKNFVSSKGYPRYCTVTTLSDGSVVLNGGRSRRGKLRGPPESREWGNALKGCDDPLFLDFLKQCLEWDPAVRMTPGQALRHPWLRRRLPKPPTGEKTSVKRITESTGAITSISKLPPPSSSASKLRTNLAQMTDANGNIQQRTVLPKLVS

SEQ ID NO: 7 - protein, 22 aa, Homo sapiens
DYPEEEQLSGAGYRVSAALEEA